SEQUENCE LISTING (253 sequences)

SEQ ID NO: 1 - protein, 6 aa, Gallus gallus
FGLSGL

SEQ ID NO: 2 - protein, 7 aa, Gallus gallus
AAVGSSL

SEQ ID NO: 3 - protein, 7 aa, Gallus gallus
TAVGSST

SEQ ID NO: 4 - protein, 6 aa, Gallus gallus
LSLSSQ

SEQ ID NO: 5 - protein, 7 aa, Gallus gallus
FGISGLG

SEQ ID NO: 6 - protein, 6 aa, Gallus gallus
PILSSF

SEQ ID NO: 7 - protein, 7 aa, Gallus gallus
SLLSQGA

SEQ ID NO: 8 - protein, 6 aa, Gallus gallus
FNLRGA

SEQ ID NO: 9 - protein, 8 aa, Gallus gallus
SAAVGSSL

SEQ ID NO: 10 - protein, 6 aa, Gallus gallus
SRVVIQ

SEQ ID NO: 11 - protein, 6 aa, Gallus gallus
SRVVLE

SEQ ID NO: 12 - protein, 8 aa, Gallus gallus
GFGISGLG

SEQ ID NO: 13 - protein, 6 aa, Gallus gallus
SRFSGR

SEQ ID NO: 14 - protein, 6 aa, Gallus gallus
RVVIQP

SEQ ID NO: 15 - protein, 8 aa, Gallus gallus
SAAVGSIL

SEQ ID NO: 16 - protein, 7 aa, Gallus gallus
IVVIGHV

SEQ ID NO: 17 - protein, 9 aa, Gallus gallus
TAVGSSTSA

SEQ ID NO: 18 - protein, 7 aa, Gallus gallus
RVVIQPS

SEQ ID NO: 19 - protein, 7 aa, Gallus gallus
SRVVIQP

SEQ ID NO: 20 - protein, 7 aa, Gallus gallus
PQGEQLQ

SEQ ID NO: 21 - protein, 9 aa, Gallus gallus
RAGAGPPSP

SEQ ID NO: 22 - protein, 7 aa, Gallus gallus
DSRVVIQ

SEQ ID NO: 23 - protein, 8 aa, Gallus gallus
PGPILSSF

SEQ ID NO: 24 - protein, 9 aa, Gallus gallus
PISSGGFGI

SEQ ID NO: 25 - protein, 8 aa, Gallus gallus
ISGLGSRF

SEQ ID NO: 26 - protein, 10 aa, Gallus gallus
SGGFGISGLG

SEQ ID NO: 27 - protein, 7 aa, Gallus gallus
SRFSGRR

SEQ ID NO: 28 - protein, 9 aa, Gallus gallus
PGLALPVLP

SEQ ID NO: 29 - protein, 8 aa, Gallus gallus
SRVVIQPS

SEQ ID NO: 30 - protein, 8 aa, Gallus gallus
PILSSFPQ

SEQ ID NO: 31 - protein, 8 aa, Gallus gallus
LGSGIFWI

SEQ ID NO: 32 - protein, 9 aa, Gallus gallus
GISGLGSRF

SEQ ID NO: 33 - protein, 9 aa, Gallus gallus
VVVTLPGPI

SEQ ID NO: 34 - protein, 8 aa, Gallus gallus
CRLSLSQP

SEQ ID NO: 35 - protein, 11 aa, Gallus gallus
SAAVGSSLSAG

SEQ ID NO: 36 - protein, 11 aa, Gallus gallus
AVGSSTSAAVG

SEQ ID NO: 37 - protein, 9 aa, Gallus gallus
VVTLPGPIL

SEQ ID NO: 38 - protein, 9 aa, Gallus gallus
PGPILSSFP

SEQ ID NO: 39 - protein, 10 aa, Gallus gallus
SAAVGSILSE

SEQ ID NO: 40 - protein, 10 aa, Gallus gallus
VPISSGGFGI

SEQ ID NO: 41 - protein, 8 aa, Gallus gallus
QLAQRNTI

SEQ ID NO: 42 - protein, 9 aa, Gallus gallus
LSGLRSNLS

SEQ ID NO: 43 - protein, 10 aa, Gallus gallus
PSPVVVTLPG

SEQ ID NO: 44 - protein, 10 aa, Gallus gallus
PPPVVVTLPG

SEQ ID NO: 45 - protein, 10 aa, Gallus gallus
ISGLGSRFSG

SEQ ID NO: 46 - protein, 9 aa, Gallus gallus
LALRQSVEA

SEQ ID NO: 47 - protein, 10 aa, Gallus gallus
PLLGPPLLGI

SEQ ID NO: 48 - protein, 10 aa, Gallus gallus
VVTLPGPILS

SEQ ID NO: 49 - protein, 8 aa, Gallus gallus
VTLEDLRR

SEQ ID NO: 50 - protein, 9 aa, Gallus gallus
PILSSFPQN

SEQ ID NO: 51 - protein, 9 aa, Gallus gallus
PILSSFPQD

SEQ ID NO: 52 - protein, 11 aa, Gallus gallus
GTVGVSAGVTR

SEQ ID NO: 53 - protein, 9 aa, Gallus gallus
FGLSRVTLN

SEQ ID NO: 54 - protein, 12 aa, Gallus gallus
TAVGSSTSAAVG

SEQ ID NO: 55 - protein, 10 aa, Gallus gallus
VVVTLPGPIL

SEQ ID NO: 56 - protein, 10 aa, Gallus gallus
PPVVVTFPGP

SEQ ID NO: 57 - protein, 10 aa, Gallus gallus
PPPVVVTFPG

SEQ ID NO: 58 - protein, 10 aa, Gallus gallus
VVIQPSPVVV

SEQ ID NO: 59 - protein, 10 aa, Gallus gallus
FGISGLGSRF

SEQ ID NO: 60 - protein, 10 aa, Gallus gallus
PGPILSSFPQ

SEQ ID NO: 61 - protein, 10 aa, Gallus gallus
FGWSGLGRGL

SEQ ID NO: 62 - protein, 9 aa, Gallus gallus
LEAQLSELR

SEQ ID NO: 63 - protein, 9 aa, Gallus gallus
IVGAWYTVR

SEQ ID NO: 64 - protein, 11 aa, Gallus gallus
LLSPPGLPGLL

SEQ ID NO: 65 - protein, 11 aa, Gallus gallus
VVVTLPGPILS

SEQ ID NO: 66 - protein, 11 aa, Gallus gallus
GFGISGLGSRF

SEQ ID NO: 67 - protein, 11 aa, Gallus gallus
PVVVTLPGPIL

SEQ ID NO: 68 - protein, 11 aa, Gallus gallus
VSQGEITVPSI

SEQ ID NO: 69 - protein, 11 aa, Gallus gallus
SILSEEGVPIS

SEQ ID NO: 70 - protein, 11 aa, Gallus gallus
PVVVTFPGPIL

SEQ ID NO: 71 - protein, 13 aa, Gallus gallus
PISSGGFGISGLG

SEQ ID NO: 72 - protein, 13 aa, Gallus gallus
VGSSTSAAVGSIL

SEQ ID NO: 73 - protein, 11 aa, Gallus gallus
GFLSSAVSLNR

SEQ ID NO: 74 - protein, 12 aa, Gallus gallus
GGFGISGLGSRF

SEQ ID NO: 75 - protein, 11 aa, Gallus gallus
PGPILSSFPQN

SEQ ID NO: 76 - protein, 11 aa, Gallus gallus
PGPILSSFPQD

SEQ ID NO: 77 - protein, 12 aa, Gallus gallus
GGFGWSGLGRGL

SEQ ID NO: 78 - protein, 12 aa, Gallus gallus
PSPVVVTLPGPI

SEQ ID NO: 79 - protein, 12 aa, Gallus gallus
VVVTLPGPILSS

SEQ ID NO: 80 - protein, 12 aa, Gallus gallus
PVVVTLPGPILS

SEQ ID NO: 81 - protein, 12 aa, Gallus gallus
SPVVVTLPGPIL

SEQ ID NO: 82 - protein, 11 aa, Gallus gallus
RVVIQPSPVVV

SEQ ID NO: 83 - protein, 11 aa, Gallus gallus
QALTEVYAKAN

SEQ ID NO: 84 - protein, 10 aa, Gallus gallus
IETLTQFLGW

SEQ ID NO: 85 - protein, 13 aa, Gallus gallus
GFGSRSLVGLGGT

SEQ ID NO: 86 - protein, 14 aa, Gallus gallus
AVGSSTSAAVGSIL

SEQ ID NO: 87 - protein, 12 aa, Gallus gallus
PVVVTFPGPILS

SEQ ID NO: 88 - protein, 14 aa, Gallus gallus
VGSSTSAAVGSILS

SEQ ID NO: 89 - protein, 13 aa, Gallus gallus
GFGISGLGSRFSG

SEQ ID NO: 90 - protein, 13 aa, Gallus gallus
SGGFGISGLGSRF

SEQ ID NO: 91 - protein, 14 aa, Gallus gallus
VPISSGGFGISGLG

SEQ ID NO: 92 - protein, 12 aa, Gallus gallus
VVIQPSPVVVTL

SEQ ID NO: 93 - protein, 13 aa, Gallus gallus
SILSEEGVPISSG

SEQ ID NO: 94 - protein, 13 aa, Gallus gallus
VMGCKAAGASRII

SEQ ID NO: 95 - protein, 13 aa, Gallus gallus
SPVVVTLPGPILS

SEQ ID NO: 96 - protein, 12 aa, Gallus gallus
SRVVIQPSPVVV

SEQ ID NO: 97 - protein, 13 aa, Gallus gallus
PSPVVVTLPGPIL

SEQ ID NO: 98 - protein, 12 aa, Gallus gallus
RVVIQPSPVVVT

SEQ ID NO: 99 - protein, 13 aa, Gallus gallus
PPPVVVTLPGPIL

SEQ ID NO: 100 - protein, 15 aa, Gallus gallus
GVPISSGGFGISGLG

SEQ ID NO: 101 - protein, 13 aa, Gallus gallus
VIQPSPVVVTLPG

SEQ ID NO: 102 - protein, 15 aa, Gallus gallus
AVGSSTSAAVGSILS

SEQ ID NO: 103 - protein, 15 aa, Gallus gallus
TAVGSSTSAAVGSIL

SEQ ID NO: 104 - protein, 14 aa, Gallus gallus
SGGFGISGLGSRFS

SEQ ID NO: 105 - protein, 13 aa, Gallus gallus
VVVTLPGPILSSF

SEQ ID NO: 106 - protein, 13 aa, Gallus gallus
PPPVVVTFPGPIL

SEQ ID NO: 107 - protein, 14 aa, Gallus gallus
GLGGFGWSGLGRGL

SEQ ID NO: 108 - protein, 13 aa, Gallus gallus
VTLPGPILSSFPQ

SEQ ID NO: 109 - protein, 14 aa, Gallus gallus
AVGSILSEEGVPIS

SEQ ID NO: 110 - protein, 14 aa, Gallus gallus
SLGLGGFGWSGLGR

SEQ ID NO: 111 - protein, 14 aa, Gallus gallus
PSPVVVTLPGPILS

SEQ ID NO: 112 - protein, 13 aa, Gallus gallus
SRVVIQPSPVVVT

SEQ ID NO: 113 - protein, 15 aa, Gallus gallus
SGGFGISGLGSRFSG

SEQ ID NO: 114 - protein, 14 aa, Gallus gallus
PPPVVVTLPGPILS

SEQ ID NO: 115 - protein, 14 aa, Gallus gallus
VAIQPPPVVVTLPG

SEQ ID NO: 116 - protein, 15 aa, Gallus gallus
PISSGGFGISGLGSR

SEQ ID NO: 117 - protein, 13 aa, Gallus gallus
PIVDVAEALLHVK

SEQ ID NO: 118 - protein, 14 aa, Gallus gallus
VVIQPSPVVVTLPG

SEQ ID NO: 119 - protein, 13 aa, Gallus gallus
RVVIQPSPVVVTL

SEQ ID NO: 120 - protein, 16 aa, Gallus gallus
TAVGSSTSAAVGSILS

SEQ ID NO: 121 - protein, 14 aa, Gallus gallus
PPPVVVTFPGPILS

SEQ ID NO: 122 - protein, 15 aa, Gallus gallus
SLGLGGFGWSGLGRG

SEQ ID NO: 123 - protein, 14 aa, Gallus gallus
VVVTLPGPILSSFP

SEQ ID NO: 124 - protein, 14 aa, Gallus gallus
PVVVTLPGPILSSF

SEQ ID NO: 125 - protein, 15 aa, Gallus gallus
AAVGSILSEEGVPIS

SEQ ID NO: 126 - protein, 15 aa, Gallus gallus
SAAVGSILSEEGVPI

SEQ ID NO: 127 - protein, 16 aa, Gallus gallus
EGVPISSGGFGISGLG

SEQ ID NO: 128 - protein, 14 aa, Gallus gallus
VVTLPGPILSSFPQ

SEQ ID NO: 129 - protein, 15 aa, Gallus gallus
PSPVVVTLPGPILSS

SEQ ID NO: 130 - protein, 16 aa, Gallus gallus
GKDGPKGVRGDAGPPG

SEQ ID NO: 131 - protein, 15 aa, Gallus gallus
SVAIQPPPVVVTLPG

SEQ ID NO: 132 - protein, 14 aa, Gallus gallus
SRVVIQPSPVVVTL

SEQ ID NO: 133 - protein, 14 aa, Gallus gallus
LHTEAAATVPDRSE

SEQ ID NO: 134 - protein, 15 aa, Gallus gallus
VVIQPSPVVVTLPGP

SEQ ID NO: 135 - protein, 14 aa, Gallus gallus
RVVIQPSPVVVTLP

SEQ ID NO: 136 - protein, 16 aa, Gallus gallus
SAAVGSILSEEGVPIS

SEQ ID NO: 137 - protein, 15 aa, Gallus gallus
PVVVTLPGPILSSFP

SEQ ID NO: 138 - protein, 15 aa, Gallus gallus
IQPSPVVVTLPGPIL

SEQ ID NO: 139 - protein, 17 aa, Gallus gallus
SVPIGAGGSLGLGGFGW

SEQ ID NO: 140 - protein, 16 aa, Gallus gallus
SLGLGGFGWSGLGRGL

SEQ ID NO: 141 - protein, 17 aa, Gallus gallus
FGGGFGGGDGILPAGEK

SEQ ID NO: 142 - protein, 17 aa, Gallus gallus
TAVGSSTSAAVGSILSE

SEQ ID NO: 143 - protein, 16 aa, Gallus gallus
PISSGGFGISGLGSRF

SEQ ID NO: 144 - protein, 19 aa, Gallus gallus
SAAVGSSLSAGSVPIGAGG

SEQ ID NO: 145 - protein, 17 aa, Gallus gallus
GVPISSGGFGISGLGSR

SEQ ID NO: 146 - protein, 15 aa, Gallus gallus
VVVTLPGPILSSFPQ

SEQ ID NO: 147 - protein, 15 aa, Gallus gallus
RVVIQPSPVVVTLPG

SEQ ID NO: 148 - protein, 15 aa, Gallus gallus
SRVVIQPSPVVVTLP

SEQ ID NO: 149 - protein, 16 aa, Gallus gallus
PSPVVVTLPGPILSSF

SEQ ID NO: 150 - protein, 16 aa, Gallus gallus
AIQPPPVVVTLPGPIL

SEQ ID NO: 151 - protein, 16 aa, Gallus gallus
IQPSPVVVTLPGPILS

SEQ ID NO: 152 - protein, 16 aa, Gallus gallus
VIQPSPVVVTLPGPIL

SEQ ID NO: 153 - protein, 17 aa, Gallus gallus
VPISSGGFGISGLGSRF

SEQ ID NO: 154 - protein, 16 aa, Gallus gallus
SRVVIQPSPVVVTLPG

SEQ ID NO: 155 - protein, 17 aa, Gallus gallus
SILSEEGVPISSGGFGI

SEQ ID NO: 156 - protein, 16 aa, Gallus gallus
PVVVTLPGPILSSFPQ

SEQ ID NO: 157 - protein, 16 aa, Gallus gallus
RVVIQPSPVVVTLPGP

SEQ ID NO: 158 - protein, 18 aa, Gallus gallus
SAAVGSILSEEGVPISSG

SEQ ID NO: 159 - protein, 16 aa, Gallus gallus
VVVTLPGPILSSFPQN

SEQ ID NO: 160 - protein, 18 aa, Gallus gallus
GVPISSGGFGISGLGSRF

SEQ ID NO: 161 - protein, 17 aa, Gallus gallus
PSPVVVTLPGPILSSFP

SEQ ID NO: 162 - protein, 17 aa, Gallus gallus
VAIQPPPVVVTLPGPIL

SEQ ID NO: 163 - protein, 17 aa, Gallus gallus
VIQPSPVVVTLPGPILS

SEQ ID NO: 164 - protein, 17 aa, Gallus gallus
VVIQPSPVVVTLPGPIL

SEQ ID NO: 165 - protein, 17 aa, Gallus gallus
SPVVVTLPGPILSSFPQ

SEQ ID NO: 166 - protein, 15 aa, Gallus gallus
VILSNTKTQYSIKIH

SEQ ID NO: 167 - protein, 17 aa, Gallus gallus
SRVVIQPSPVVVTLPGP

SEQ ID NO: 168 - protein, 17 aa, Gallus gallus
DSRVVIQPSPVVVTLPG

SEQ ID NO: 169 - protein, 17 aa, Gallus gallus
PVVVTLPGPILSSFPQN

SEQ ID NO: 170 - protein, 17 aa, Gallus gallus
RVVIQPSPVVVTLPGPI

SEQ ID NO: 171 - protein, 19 aa, Gallus gallus
GVPISSGGFGISGLGSRFS

SEQ ID NO: 172 - protein, 18 aa, Gallus gallus
SVAIQPPPVVVTLPGPIL

SEQ ID NO: 173 - protein, 18 aa, Gallus gallus
VAIQPPPVVVTLPGPILS

SEQ ID NO: 174 - protein, 18 aa, Gallus gallus
VVIQPSPVVVTLPGPILS

SEQ ID NO: 175 - protein, 18 aa, Gallus gallus
PSPVVVTLPGPILSSFPQ

SEQ ID NO: 176 - protein, 20 aa, Gallus gallus
GVPISSGGFGISGLGSRFSG

SEQ ID NO: 177 - protein, 18 aa, Gallus gallus
AVPLVRPVTMVPSIPGIP

SEQ ID NO: 178 - protein, 18 aa, Gallus gallus
PPPVVVTLPGPILSSFPQ

SEQ ID NO: 179 - protein, 18 aa, Gallus gallus
SPVVVTLPGPILSSFPQN

SEQ ID NO: 180 - protein, 18 aa, Gallus gallus
LPGPGLQLLHGTTTLAFK

SEQ ID NO: 181 - protein, 18 aa, Gallus gallus
PPPVVVTFPGPILSSFPQ

SEQ ID NO: 182 - protein, 19 aa, Gallus gallus
SVAIQPPPVVVTLPGPILS

SEQ ID NO: 183 - protein, 19 aa, Gallus gallus
SSVAIQPPPVVVTLPGPIL

SEQ ID NO: 184 - protein, 18 aa, Gallus gallus
RVVIQPSPVVVTLPGPIL

SEQ ID NO: 185 - protein, 19 aa, Gallus gallus
PSPVVVTLPGPILSSFPQN

SEQ ID NO: 186 - protein, 21 aa, Gallus gallus
SILSEEGVPISSGGFGISGLG

SEQ ID NO: 187 - protein, 19 aa, Gallus gallus
SRVVIQPSPVVVTLPGPIL

SEQ ID NO: 188 - protein, 19 aa, Gallus gallus
RVVIQPSPVVVTLPGPILS

SEQ ID NO: 189 - protein, 22 aa, Gallus gallus
SVPIGAGGSLGLGGFGWSGLGR

SEQ ID NO: 190 - protein, 22 aa, Gallus gallus
TAVGSSTSAAVGSILSEEGVPI

SEQ ID NO: 191 - protein, 22 aa, Gallus gallus
SAAVGSILSEEGVPISSGGFGI

SEQ ID NO: 192 - protein, 21 aa, Gallus gallus
SEEGVPISSGGFGISGLGSRF

SEQ ID NO: 193 - protein, 20 aa, Gallus gallus
SRVVIQPSPVVVTLPGPILS

SEQ ID NO: 194 - protein, 23 aa, Gallus gallus
SVPIGAGGSLGLGGFGWSGLGRG

SEQ ID NO: 195 - protein, 20 aa, Gallus gallus
DSRVVIQPSPVVVTLPGPIL

SEQ ID NO: 196 - protein, 23 aa, Gallus gallus
TAVGSSTSAAVGSILSEEGVPIS

SEQ ID NO: 197 - protein, 22 aa, Gallus gallus
LRGDVGPVGRTGEQGIAGPPGF

SEQ ID NO: 198 - protein, 21 aa, Gallus gallus
SRVVIQPSPVVVTLPGPILSS

SEQ ID NO: 199 - protein, 21 aa, Gallus gallus
VVIQPSPVVVTLPGPILSSFP

SEQ ID NO: 200 - protein, 22 aa, Gallus gallus
GVPISSGGFGISGLGSRFSGRR

SEQ ID NO: 201 - protein, 24 aa, Gallus gallus
SVPIGAGGSLGLGGFGWSGLGRGL

SEQ ID NO: 202 - protein, 21 aa, Gallus gallus
DSRVVIQPSPVVVTLPGPILS

SEQ ID NO: 203 - protein, 21 aa, Gallus gallus
VIQPSPVVVTLPGPILSSFPQ

SEQ ID NO: 204 - protein, 24 aa, Gallus gallus
AVGSILSEEGVPISSGGFGISGLG

SEQ ID NO: 205 - protein, 21 aa, Gallus gallus
RVVIQPSPVVVTLPGPILSSF

SEQ ID NO: 206 - protein, 23 aa, Gallus gallus
SILSEEGVPISSGGFGISGLGSR

SEQ ID NO: 207 - protein, 21 aa, Gallus gallus
IPSSVPESSFKIKGNSSSFHI

SEQ ID NO: 208 - protein, 22 aa, Gallus gallus
VAIQPPPVVVTLPGPILSSFPQ

SEQ ID NO: 209 - protein, 25 aa, Gallus gallus
TAVGSSTSAAVGSILSEEGVPISSG

SEQ ID NO: 210 - protein, 22 aa, Gallus gallus
VVIQPSPVVVTLPGPILSSFPQ

SEQ ID NO: 211 - protein, 23 aa, Gallus gallus
PSPVVVTLPGPILSSFPQNTAVG

SEQ ID NO: 212 - protein, 20 aa, Gallus gallus
KENFEVLCKDGTRKPVTDAE

SEQ ID NO: 213 - protein, 21 aa, Gallus gallus
SIQACKLAQSHGWGVMVSHRS

SEQ ID NO: 214 - protein, 22 aa, Gallus gallus
SRVVIQPSPVVVTLPGPILSSF

SEQ ID NO: 215 - protein, 22 aa, Gallus gallus
RVVIQPSPVVVTLPGPILSSFP

SEQ ID NO: 216 - protein, 23 aa, Gallus gallus
EQPPLCVASVSVKEPASETPAVP

SEQ ID NO: 217 - protein, 23 aa, Gallus gallus
SVAIQPPPVVVTLPGPILSSFPQ

SEQ ID NO: 218 - protein, 26 aa, Gallus gallus
SAAVGSILSEEGVPISSGGFGISGLG

SEQ ID NO: 219 - protein, 24 aa, Gallus gallus
SILSEEGVPISSGGFGISGLGSRF

SEQ ID NO: 220 - protein, 24 aa, Gallus gallus
PSPVVVTLPGPILSSFPQNTAVGS

SEQ ID NO: 221 - protein, 23 aa, Gallus gallus
VVIQPSPVVVTLPGPILSSFPQN

SEQ ID NO: 222 - protein, 23 aa, Gallus gallus
SRVVIQPSPVVVTLPGPILSSFP

SEQ ID NO: 223 - protein, 24 aa, Gallus gallus
SSVAIQPPPVVVTLPGPILSSFPQ

SEQ ID NO: 224 - protein, 23 aa, Gallus gallus
RVVIQPSPVVVTLPGPILSSFPQ

SEQ ID NO: 225 - protein, 25 aa, Gallus gallus
SILSEEGVPISSGGFGISGLGSRFS

SEQ ID NO: 226 - protein, 27 aa, Gallus gallus
TSAAVGSILSEEGVPISSGGFGISGLG

SEQ ID NO: 227 - protein, 26 aa, Gallus gallus
SILSEEGVPISSGGFGISGLGSRFSG

SEQ ID NO: 228 - protein, 24 aa, Gallus gallus
SRVVIQPSPVVVTLPGPILSSFPQ

SEQ ID NO: 229 - protein, 28 aa, Gallus gallus
TAVGSSTSAAVGSILSEEGVPISSGGFG

SEQ ID NO: 230 - protein, 28 aa, Gallus gallus
STSAAVGSILSEEGVPISSGGFGISGLG

SEQ ID NO: 231 - protein, 24 aa, Gallus gallus
RVVIQPSPVVVTLPGPILSSFPQN

SEQ ID NO: 232 - protein, 25 aa, Gallus gallus
SRVVIQPSPVVVTLPGPILSSFPQN

SEQ ID NO: 233 - protein, 25 aa, Gallus gallus
DSRVVIQPSPVVVTLPGPILSSFPQ

SEQ ID NO: 234 - protein, 29 aa, Gallus gallus
TAVGSSTSAAVGSILSEEGVPISSGGFGI

SEQ ID NO: 235 - protein, 29 aa, Gallus gallus
SAAVGSILSEEGVPISSGGFGISGLGSRF

SEQ ID NO: 236 - protein, 24 aa, Gallus gallus
VEQLVNLQHLDVAYNLLLEHAQLA

SEQ ID NO: 237 - protein, 28 aa, Gallus gallus
SVAIQPPPVVVTLPGPILSSFPQSTAVG

SEQ ID NO: 238 - protein, 28 aa, Gallus gallus
SILSEEGVPISSGGFGISGLGSRFSGRR

SEQ ID NO: 239 - protein, 30 aa, Gallus gallus
TSAAVGSILSEEGVPISSGGFGISGLGSRF

SEQ ID NO: 240 - protein, 29 aa, Gallus gallus
SSVAIQPPPVVVTLPGPILSSFPQSTAVG

SEQ ID NO: 241 - protein, 31 aa, Gallus gallus
STSAAVGSILSEEGVPISSGGFGISGLGSRF

SEQ ID NO: 242 - protein, 31 aa, Gallus gallus
PSPVVVTLPGPILSSFPQNTAVGSSTSAAVG

SEQ ID NO: 243 - protein, 33 aa, Gallus gallus
TAVGSSTSAAVGSILSEEGVPISSGGFGISGLG

SEQ ID NO: 244 - protein, 31 aa, Gallus gallus
AVGSILSEEGVPISSGGFGISGLGSRFSGRR

SEQ ID NO: 245 - protein, 27 aa, Gallus gallus
NKVLVHCAMGRSRSATLVLAYLMIYKN

SEQ ID NO: 246 - protein, 32 aa, Gallus gallus
SSVAIQPPPVVVTLPGPILSSFPQSTAVGSTA

SEQ ID NO: 247 - protein, 32 aa, Gallus gallus
AAVGSILSEEGVPISSGGFGISGLGSRFSGRR

SEQ ID NO: 248 - protein, 35 aa, Gallus gallus
TAVGSSTSAAVGSILSEEGVPISSGGFGISGLGSR

SEQ ID NO: 249 - protein, 33 aa, Gallus gallus
SAAVGSILSEEGVPISSGGFGISGLGSRFSGRR

SEQ ID NO: 250 - protein, 34 aa, Gallus gallus
PSPVVVTLPGPILSSFPQNTAVGSSTSAAVGSIL

SEQ ID NO: 251 - protein, 35 aa, Gallus gallus
PSPVVVTLPGPILSSFPQNTAVGSSTSAAVGSILS

SEQ ID NO: 252 - protein, 36 aa, Gallus gallus
TAVGSSTSAAVGSILSEEGVPISSGGFGISGLGSRF

SEQ ID NO: 253 - protein, 29 aa, Gallus gallus
RPRVYLHCTNPRHPKYREGDVTLFALNLS

(19) United States
(12) Patent Application Publication
(10) Pub. No.: US 2025/0160325 A1
(43) Pub. Date: May 22, 2025

(54) METHOD OF USING/APPLYING A KERATIN HYDROLYSIS PEPTIDE SOLUTION TO IMPROVE THE DROUGHT STRESS TOLERANCE OF CORN

(71) Applicant: CH Biotech R&D Co., Ltd., Nantou City (TW)

(72) Inventors: Kai XIA, Nantou (TW); Ya-Wen HUANG, Taichung (TW); Gui-Jun LI, Taichung (TW)

(73) Assignee: CH Biotech R&D Co., Ltd., Nantou City (TW)

(21) Appl. No.: 18/642,007

(22) Filed: Apr. 22, 2024

(30) Foreign Application Priority Data
Nov. 17, 2023 (TW) 112144619

(51) Int. Cl. A01N 37/46; A01P 21/00; C12P 21/06

(52) U.S. Cl. CPC A01N 37/46; A01P 21/00; C12P 21/06

(57) ABSTRACT

Present invention teaches the method of using a keratin hydrolysis peptide (“KHP”) solution to improve the corn plant's drought tolerance and ultimate production yield in hot weather. By selectively choosing specific weights of feathers and water, and treating the mixture to a high-temperature high-pressure hydrolysis process, the resulting solution is confirmed to contain at least 253 peptides and then infused to the soil in which the corn seeds and/or seedlings are planted. Optionally, the KHP solution can be diluted by water, as disclosed in the specification, for applying to the soil.

### PRIORITY CLAIM TO FOREIGN APPLICATION

Applicant hereby makes priority claim to a Taiwan application, number 112144619, having the Taiwan filing date of Nov. 17 2023.

### SEQUENCE LISTING/INCORPORATION BY REFERENCE

Table I (in Sequence Listing XML format) shows the at least 253 peptides and its annotated sequences for the solution generated in accordance with the disclosure of this application. The Sequence Listing XML file complies with the WIPO ST.26 requirements. Said XML copy, created on Mar. 17, 2024, is named Table-I-253_sequence and is 216 bytes in size.

Applicant hereby incorporates by reference said Sequence Listing XML file in its entirety as part of the disclosure and specification of the present application.

### BACKGROUND OF THE INVENTION

Present invention disclosed and claimed the method and application of a keratin hydrolysis peptide (“KHP”) solution for improving the drought tolerance of corn plants, by using the thermally hydrolyzed feather keratin peptide solution to the soil where the corn seedlings are transplanted in. The application of the KHP solutions as taught herein contributed to the drought tolerance of corn seedlings, reduced and minimized the injuries and growth damages suffered by corn plants due to drought conditions, and ultimately improved the overall production yield of corn.

The KHP solution is made by a hydrolysis process using feathers and water, via a high-temperature and high-pressure process, resulting in a solution that has many beneficial applications in the fields of horticulture, agriculture and potentially other farming businesses.

Hydrolyzed keratin has long been used to strengthen hairs, reduce hair splitting and breakage. Other beneficial uses include skin moisturization and wound healing. Keratin hydrolysate has also been known to function as a biofertilizer, boosting plants' growth by enhancing the plants' ability to receive and utilize nutrients, including commonly applied fertilizers.

The corn crops originated from South America, primarily in the tropical regions, with good adaptability to different weather patterns and good for growing in warm, moist areas with ample sun lights. The short growth period of the corn plants makes them good for all-season planting and harvesting.

The recent climate changes have great impacts on agriculture efficiency and production globally. Specifically, the drought conditions brought on by the climate substantially changes affects the corn production. When ambient temperature rises above 35° C., plants' ability to bloom and flower will decrease; when ambient temperature rises above 35° C., the male spikes (stamens) in flowers no longer open up and the pollination to the female spikes (stigmas) can hardly happen. Sustained period of high temperature and drought certainly results in more damages to corns' growth and production.

In the corn agriculture, farmers have come up with different options to deal with the high temperature and drought conditions, such as selecting drought-tolerant species and reduce the growth intensity of corn plants to provide better water accessibility, addition to the ever present fertilizations used to the add nutrients to the soil.

A published Chinese patent CN11116656698A presented a corn gene Zm0001d018037 that is suitable for leaflet crops. An European patent EP2713751A1 disclosed a cultivation style without water where certain plant species can withstand tough drought condition when selected agri-chemical compounds are used in the farming process, resulting in crop production without water irrigation. Another published patent CN111700073A provides a combination mechanism of a seedling with certain accompany agents that help with the drought tolerance of the plants.

However, the technologies stated above are not helpful to the corn-drought problems as the gene, the no-water growing and the seedling combination method are all inapplicable to corns.

The inventors of present application, through their research and experiment, discovered and confirmed that the application of the KHP solution as taught herein is the best and cost-effective way of ameliorating the harms and damages due to the drought conditions.

### SUMMARY OF THE INVENTION

The keratin solution is primarily based upon feather, which contains 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by the mixture of α-amylase and protease to hydrolyze feather waste, resulting in a mixture of amino acids, fatty acids, and sugars. Nurdiawati experimented and adopted certain specific high-temperature and high-pressure setting in the hydrolysis process and discovered that the resulting solution, when mixed with some potassium and other minerals, can boost the growth of Pogostemon cablin and Vigna radiata, as reported in International Journal of Recycling or Organic Waste in Agriculture (8:221-232, 2019).

The inventors of present application, under the aegis of CH Biotech, developed and selected different feather and water compositions to perform the hydrolysis at higher temperature and higher-pressure setting, resulting with different keratin hydrolysis peptide (“KHP”) solution that can be used on different crops/plants.

The selected embodiment of present invention uses a mixture of water and feathers, and subject the mixture to a thermal hydrolysis process to create KHP solutions based upon temperature/pressure parameters as noted below.

The inventors used the Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The solutions are infused to the soil containing the corn seedlings. The solutions can be diluted with water at ratio of 50-1,000 for applying to the corn seedlings. The preferred range of dilution ratio is between 50-100.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

FIG. 1 shows the comparison of root's dry weights among the Normal group vis-à-vis the 3 drought-plagued groups, consisting of a Compare group and 2 groups applied with KHP-1 50× and KHP-1 100× solutions.

FIG. 2A shows the comparison of corn plant heights among the Normal group vis-à-vis the 3 drought-plagued groups, consisting of a Compare group and 2 groups applied with KHP-1 50× and KHP-1 100× solutions.

FIG. 2B shows the comparison of corn plant's above-ground stem widths among the Normal group vis-à-vis the 3 drought-plagued groups, consisting of a Compare group and 2 groups applied with KHP-1 50× and KHP-1 100× solutions.

FIG. 2C shows the comparison of the corn plant's above-ground bio mass fresh weights among the Normal group vis-à-vis the 3 drought-plagued groups, consisting of a Compare group and 2 groups applied with KHP-1 50× and KHP-1 100× solutions.

FIG. 2D shows the comparison of the corn plant's above-ground bio mass dry weights among the Normal group vis-à-vis the 3 drought-plagued groups, consisting of a Compare group and 2 groups applied with KHP-1 50× and KHP-1 100× solutions.

FIG. 3 shows comparisons of chlorophyll counts among the Normal group vis-à-vis the 3 drought-plagued groups, consisting of a Compare group and 2 groups applied with KHP-1 50× and KHP-1 100× solutions. Based upon the SPAD measurements taken from the 4^{th}, 5^{th} and 6^{th} leaves, the results are shown in three sets noted as A, B and C respectively.

### DETAILED DESCRIPTION OF THE INVENTION

The keratin hydrolysis peptide (“KHP”) solution of present invention is made by a high-temperature and high-pressure process to treat a mixture of water and feathers as shown in the parameters herein.

The mixture ratio, temperature, pressure and duration parameters are shown herein:

| | | | Water content | | | | | |
|---|---|---|---|---|---|---|---|---|
| | Feather | Water | in feather | Pressure | Temp. | Time | Mass | Concen. |
| | (kg) | (kg) | (%) | (kg/cm²) | (° C.) | (min) | (Da) | (ppm) |
| | 66 | 44 | 50% | 16 | 195 | 40 | 593.3~3828.0 | 200000 |
| KHP-1 | 50 | 40 | 50% | 12 | 185 | 80 | 593.3~3508.9 | 301500 |
| | 70 | 0 | 46% | 13 | 180 | 40 | 705.9~3194.7 | 381250 |

The hydrolysis process, in the embodiment noted as KHP-1 takes the steps of:
a. Preparing the KHP solution by mixing 50 kg of feathers whose content is 50% water with 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm.

The confirmation of some of the 253 peptides is further done by referencing the BIOPEP-UWM database.

The KHP solution can then be applied to the soil containing the corn seeds or seedlings during the early growth stage. For purpose of this invention, the specific KHP solution, based upon the weight combination of 50 kg feathers and 40 kg water, is named as KHP-1.

Generally, for soil infusion, the dilution ratios are between 50-1,000, with 50-100 being the preferred range. The notation of 50× or 100× indicates the dilution ratio is 50 times, or 100 times.

To confirm the effectiveness of applying the KHP solution as taught herein, the inventors set up four (4) groups of corn plants: Normal, Compare, KHP-1 50× and KHP-1 100×.

The 4 groups of corn plants are planted from seeds in fertile top soil about 2 cm deep in pots sized about 3.5 inches, and set a growth temperature of 23° C.-25° C.

Normal Group corn seedlings are given water in a normal growing pattern, no interruption of water supply. The other three (3) groups' corn seedlings are given drought treatment: 20 days without water. On the second day after the watering is resumed, each pots is given 50 ml of liquid, with the liquid being water only in the Compare group and the other two group given KHP-1 50× or KHP-1 100× solutions. Various measurements are then taken to assess the effectiveness of KHP solutions' ameliorating power, with 50× and 100× dilution ratios, to the drought-plagued groups.

For simplicity, the groupings are shown herein:

| | | Applying | | |
|---|---|---|---|---|
| Group | Setting | style | Solution | Dilution Ratio |
| 1 | Normal water | Soil infusion | water | — |
| 2 | Drought | | | — |
| 3 | | | KHP | 50x |
| 4 | | | | 100x |

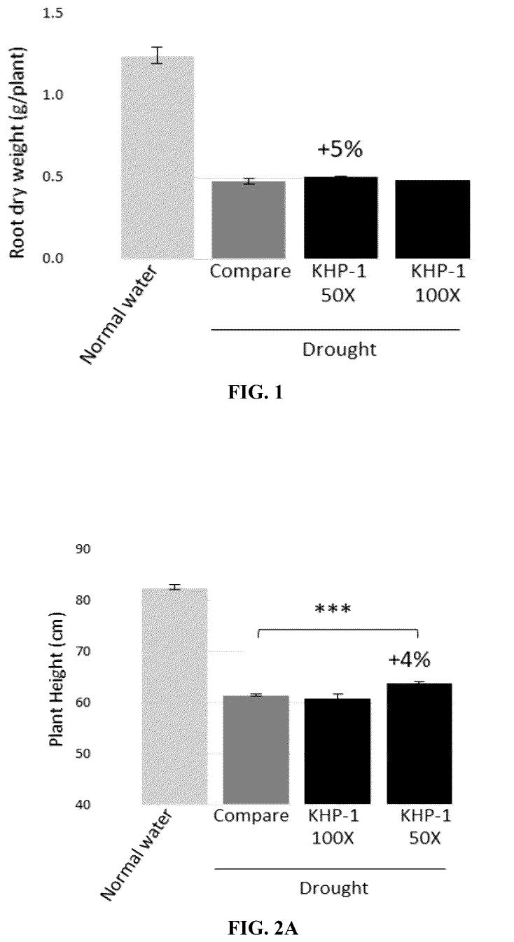

The ameliorating effect of the KHP-1 solutions is shown by measuring the roots' dry weights. As shown in FIG. 1, the Compare group's dry weight fell substantially from the Normal group. But the KHP-1 50× group (having been applied the KHP-1 50 solution) shows 5% increase over that of the Compare group.

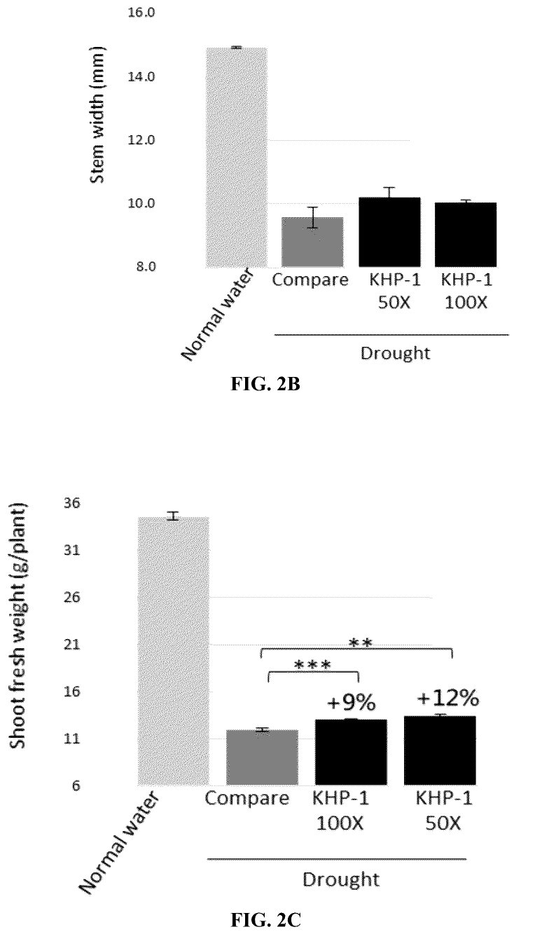

In terms of the growth heights, the KHP-1 50 group shows 4% increase over the Compare group that went through the same drought (20 days), as reflected in FIG. 2A.

FIG. 2B shows the stem width comparison of the corn plant's above-ground portion. Bot KHP-1 50× and KHP-1 100× groups show improvements over the Compare group.

FIG. 2C shows the comparison of the corn plant's above-ground bio mass fresh weights among the Normal group vis-à-vis the 3 drought-plagued groups. The two KHP-1 groups, KHP-1 50× and KHP-1 100×, show, respectively, 9% and 12% improvement over that of the Compare group.

FIG. 2D shows the comparison of the corn plant's above-ground bio mass dry weights among the Normal group vis-à-vis the 3 drought-plagued groups. The two KHP-1 groups, KHP-1 50× and KHP-1 100×, show, respectively, 5% and 11% improvement over that of the Compare group.

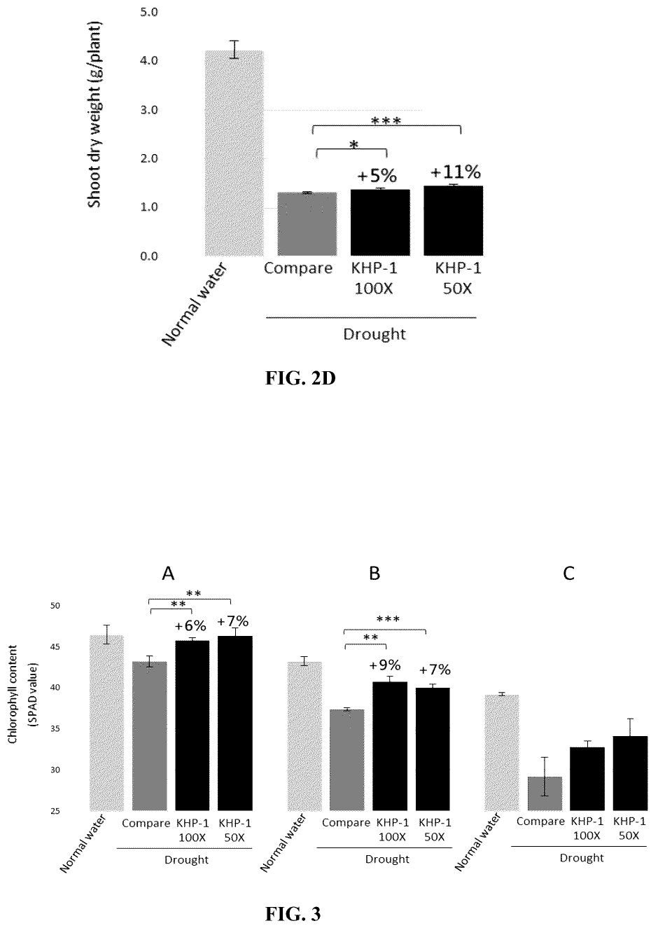

To test the KHP-1 solution's ameliorating effect on corn plant's photosynthesis capacities, the inventors extract the 4^{th}, 5^{th} and 6^{th} leaves from the corn plant on the second day after a 20-day drought, and use a SPAD 502 Plus to measure the chlorophyll counts. The results are tabulated in FIG. 3; the three sub-sets A, B and C correspond to the SPA readings of the 4^{th}, 5^{th} and 6^{th} leaves respectively.

As can be seen in FIG. 3, the SPAD readings in the two KHP-1 solution groups contain higher chlorophyll counts than that of the drought (Compare) group, in each of the A, B or C sub-sets, proving the effectiveness of the KHP-1 solutions to reduce the damages and harms suffered by corn plants due to drought conditions.

As has been proven by the tests and the scientific analysis/measurement taken by the inventors, the method of creating the KHP solution and the method of application to the corn seedling will help with the lessening of injuries suffered by corn in drought conditions and contribute to overall higher production yield of corns.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. A method of using a keratin hydrolysis peptide (KHP) solution to improve the drought tolerance of corn plants, comprising the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. infusing the solution to the soil containing the corn seeds or seedlings.

2. The method of using a keratin hydrolysis peptide solution of claim 1 wherein the solution is diluted with water by volume at the ratio of 1:50-1,000 for infusing to the soil containing the corn seeds or seedlings.

3. The method of using a keratin hydrolysis peptide (KHP) solution of claim 1 wherein the solution is diluted with water by volume at the ratio of 1:50-100 for infusing to the soil containing the corn seeds or seedlings.
